(19) United States
(12) Patent Application Publication
(10) Pub. No.: US 2026/0345518 A1
(43) Pub. Date: Nov. 5, 2026

(54) NON-INVASIVE DIMETHYL TRISULFIDE FORMULATIONS SUITABLE FOR TREATMENT OF CYANIDE POISONING

(71) Applicant: Southwest Research Institute, San Antonio, TX (US)

(72) Inventor: Hong DIXON, Helotes, TX (US)

(21) Appl. No.: 18/772,394

(22) Filed: Jul. 15, 2024

Related U.S. Application Data
(60) Provisional application No. 63/516,025, filed on Jul. 27, 2023.

(51) Int. Cl. A61K 31/105; A61K 9/00; A61K 47/10; A61K 47/14; A61K 47/22; A61K 47/26

(52) U.S. Cl. CPC A61K 31/105; A61K 9/007; A61K 47/10; A61K 47/14; A61K 47/22; A61K 47/26

(57) ABSTRACT

A non-invasive dimethyl trisulfide formulation (DMTS). The formulation shows efficacy for treatment of cyanide intoxication via intranasal administration. The compositions also show particularly useful stability such that the DMTS remains stable for therapeutic use over extended time periods.

### STATEMENT REGARDING FEDERALLY SPONSORED RESEARCH OR DEVELOPMENT

This invention was made with government support under Contract Number W81XWH22C0097, awarded by the U.S. Army Medical Research Acquisition Activity under the USA Medical Research Institute, Chemical Defense. The government has certain rights in the invention.

### FIELD

The present invention stands directed at non-invasive dimethyl trisulfide formulation (DMTS). The formulation shows efficacy for treatment of cyanide intoxication via intranasal administration. The compositions also show particularly useful stability such that the DMTS remains stable for therapeutic use over extended time periods.

### BACKGROUND

Dimethyl trisulfide formulations have been reported for use in the treatment of cyanide intoxication. For example, U.S. Publ. 2020/0345658 reports on DMTS compositions that are particularly suitable for use in autoinjectors. However, a need remains to provide DMTS formulations as a viable drug suitable for use to treat cyanide intoxication and suitable for a non-invasive route for delivery, such as intranasal administration, along with improved stability.

### SUMMARY

A non-invasive dimethyl trisulfide formulation comprising dimethyl trisulfide at a level of 15.0% (wt.) to 80.0% (wt.), maltoside based non-ionic detergent at a level of 0.1% (wt.) to 5.0% (wt.), organic alcohol at a level of 5.0% (wt.) to 65.0% (wt.), and Vitamin E or a Vitamin E derivative at a level of 0.1 wt. % to 50.0 wt. %.

An intranasal delivery device containing a dimethyl trisulfide formulation, wherein the formulation comprises dimethyl trisulfide at a level of 15.0% (wt.) to 80.0% (wt.), maltoside based non-ionic detergent at a level of 0.1% (wt.) to 5.0% (wt.), organic alcohol at a level of 5.0% (wt.) to 65.0% (wt.), and Vitamin E or a Vitamin E derivative at a level of 0.1 wt. % to 50.0 wt. %.

A method for intranasal delivery of a dimethyl trisulfide formulation comprising providing a dimethyl trisulfide formulation comprising dimethyl trisulfide at a level of 15.0% (wt.) to 80.0% (wt.), maltoside based non-ionic detergent at a level of 0.1% (wt.) to 5.0% (wt.), organic alcohol at a level of 5.0% (wt.) to 65.0% (wt.); and Vitamin E or a Vitamin E derivative at a level of 0.1 wt. % to 50.0 wt. %. This is then followed by incorporating the dimethyl trisulfide formulation into a intranasal delivery device and delivering the dimethyl trisulfide formulation into a nasal cavity.

### BRIEF DESCRIPTION OF THE DRAWINGS

FIG. 1A shows the respiratory epithelium after 3-days post dosing with a placebo formulation.

FIG. 1B shows the olfactory epithelium after 3-days post dosing with a placebo formulation.

FIG. 2A shows the respiratory epithelium after 3-days post dosing with formulation F4.

FIG. 2B shows the olfactory epithelium after 3-days posting dosing with formulation F4.

FIG. 3A shows the respiratory epithelium after 4 weeks post dosing with formulation F4.

FIG. 3B shows the olfactory epithelium after 4 weeks post dosing with formulation F4.

FIG. 4 shows the use of neat DMTS dosing and the results after 3-days post dosing on the olfactory epithelium.

### DETAILED DESCRIPTION

The present invention stands directed at a DMTS formulation for non-invasive drug administration. Preferably, the formulation comprises, consists essentially of, or consists of DMTS in combination with a water soluble maltoside based nonionic detergent composed of a maltoside sugar and alkyl side chain, in the presence of an organic alcohol (e.g., ethanol, propanol, isopropanol, 2-(2-ethoxyethoxy)ethanol, propylene glycol or 2-(2-ethoxyethoxy)ethanol (C₆H₁₄O₃) which is sold under the trademark Transcutol HP, along with Vitamin E or Vitamin E derivatives. Preferably, the DMTS is present at a level of 15.0% (wt.) to 80.0% (wt.), including all values and increments therein. More preferably, the level of DMTS may be 50.0% (wt.) to 80.0% (wt.). The water soluble maltoside based nonionic detergent is present at a level of 0.1% (wt.) to 5.0% (wt.), including all values and increments therein. The organic alcohol is present at a level of 5.0% (wt.) to 65.0% (wt.) including all values and increments therein. The Vitamin E or Vitamin E derivative is present at a level of 0.1 wt. % to 50.0 wt. %, including all values and increments therein. The sum of the ingredients in a given formulation is therefore selected to add up to 100%.

The composition described above may also optionally include other ingredients. For example, a poly(alkylene glycol), such as poly(ethylene glycol). The poly(alkylene glycol) may therefore be present at a level of 5.0% (wt.) to 40.0% (wt.), including all values and increments therein. Reference to a poly(alkylene glycol) is reference to polymers of ethylene oxide. The polyethylene glycol preferably has a molecular weight of 300 to 1000. Another optional ingredient is water, which may also be present at a level of 5.0% (wt.) to 40.0% (wt.). A still further optional ingredient is a glycerol polyethylene glycol ricinoleate and sold under the tradename Kolliphor ELP, which may again be present at a level of 5.0% (wt.) to 40.0% (wt.). Finally, one may also include ethyl laurate at a level of 5.0% (wt.) to 40.0% (wt.). Again, if optional ingredients are selected, the sum of the ingredients in a given formulation is selected to reach 100%.

Reference to a Vitamin E derivative is reference to those derivatives of Vitamin E that function to increase the relative hydrophilic character of Vitamin E. Some examples include tocopheryl acetate, tocopheryl glucoside and tocopheryl phosphate. The maltoside based non-ionic detergent is reference herein to a hydrophilic maltose head and a hydrophobic alkyl chain. For example, a maltoside based non-ionic detergent as set out in the formula below wherein the R group preferably has 8 to 15 carbon atoms. Such carbon atoms are therefore those that contain the requisite hydrogen substitution to provide the hydrophobic alkyl chain functionality. Particularly preferred maltosides therefore include at least one of octyl maltoside, nonyl maltoside, undecyl maltoside or dodecyl maltoside.

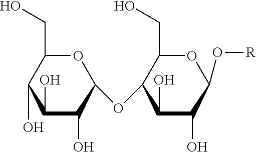

The above referenced formulations have been identified as particularly suitable for non-invasive drug administration such as intranasal delivery. The intranasal delivery herein is preferably achieved vis the use of a commercially available intranasal device. One particular suitable nasal atomizer includes the MAD Nasal Intranasal Mucosal Atomization Device distributed by Teleflex.

Table 1 below identifies the non-limiting exemplary and preferred non-invasive dimethyl trisulfide formulations herein (Examples 2-4) Example 1 did not include Vitamin E.

TABLE 1
Preferred Non-Invasive DMTS Formulations

| Example | DMTS formulation | Composition (wt %) |
|---|---|---|
| 1 | ~15% formulation #1 | 1.0% dodecyl maltoside, 6.5% |
| | (F1) | ethanol, 12.1% ethyl laurate, 12.8% |
| | | water, 17.0% DMTS, 24.8% |
| | | Kolliphor ELP, 25.8% Propylene |
| | | glycol |
| 2 | ~15% formulation #2 | 0.99% dodecyl maltoside, 17.9% |
| | (F2) | DMTS, 18.9% vitamin E, 62.7% |
| | | ethanol |
| 3 | ~50% formulation | 0.2% dodecyl maltoside, 9.6% |
| | (F3) | vitamin E, 17.7% ethanol, 23.1% |
| | | Transcutol HP, 49.3% DMTS |
| 4 | ~80% formulation | 0.3% dodecyl maltoside, 10.6% |
| | (F4) | vitamin E, 12.3% ethanol, 76.8% |
| | | DMTS |

Table 2 below sets for a stability study for Example 4 from Table 1. The stability of Example 4 was evaluated by monitoring the concentration of DMTS in the formulation over a period of four (4) months at ambient temperature in sealed (capped) vial. It is therefore contemplated that the formulations herein can remain stable within an intranasal device and remain effective for delivery, for a period of up to 6-9 months.

TABLE 2
Stability Evaluation

| DMTS concentration | |
|---|---|
| (mg/mL) | Comments |
| 767 | T0 |
| 742 | 4-month stored at room temperature, 97% |
| | assay of T0 value |

### Efficacy Examples

The formulations were shipped to U.S. Army Medical Research Institute of Chemical Defense (USAMRICD) for testing. An antidote potency ratio (APR) of each formulation was determined, which is the LD₅₀ of cyanide treatment with antidote divided by the LD₅₀ of cyanide treatment without antidote. A volume of 60 μL was delivered in the rodent study. As shown in Table 3 below, the APR were 1.3, 2, 2.8, and 3.67 for the four formulations respectively. Further study showed the APR of 3.67, and 4.3 for male and female CD rats respectively (Table 4). These results show that comparable efficacy of the intranasal formulation compared to the previously developed intramuscular injection formulation.

TABLE 3
Antidote Potency Ratios
Intranasal DMTS Efficacy

| | DMTS | Placebo | DMTS | Placebo | DMTS | Placebo | DMTS | Placebo |
|---|---|---|---|---|---|---|---|---|
| Volume | 15% | Vehicle | 15% | Vehicle | 50% | Vehicle | 80% | Vehicle |
| 60 μl | F1 | F1 | F2 | F2 | F3 | F3 | F4 | F4 |
| KCN LD | 14 | 10.5 | 25.8 | 12.3 | 31.72 | 12.8 | 48.75 | 13.1 |
| 50 | | | | | | | | |
| APR | 1.3 | — | 2 | — | 2.8 | — | 3.67 | — |

TABLE 4
Antidote Potency Ratio
Intranasal DMTS Efficacy- 80%

| | | |
|---|---|---|
| Volume 60 μl | Male CD Rats | Female CD Rats |

| | | | | |
|---|---|---|---|---|
| KCN LD 50 | 47.85 | 13.04 | 62 | 14.4 |
| APR | 3.67 | — | 4.3 | — |

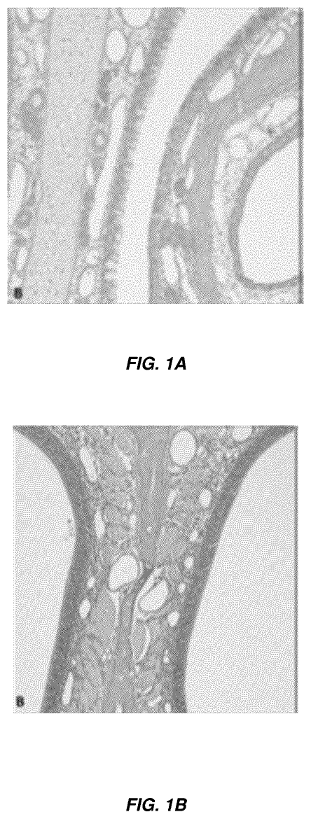

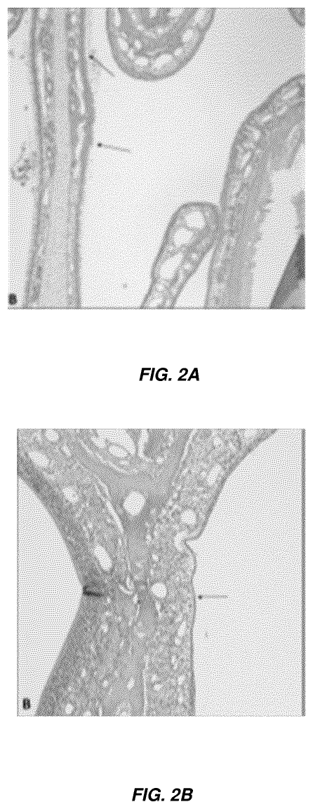

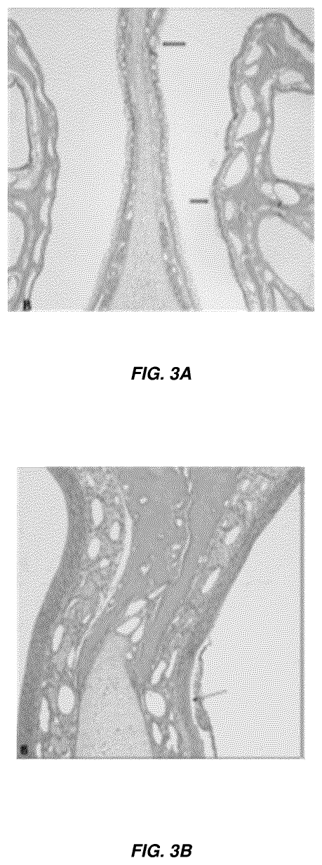

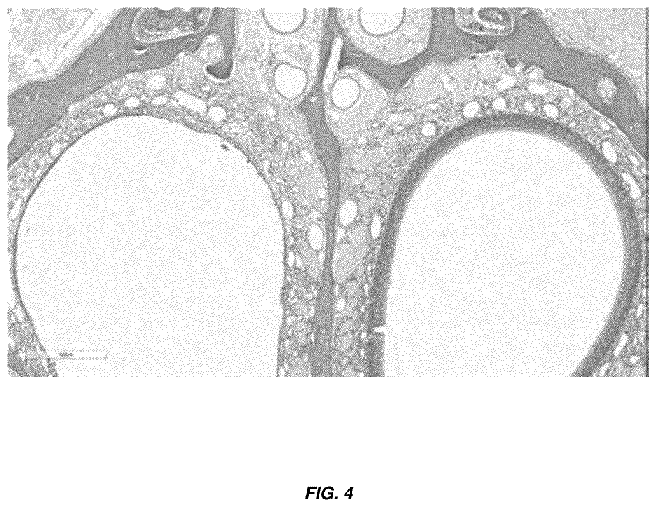

Next, pictures of representative stained tissues after dosing, as compared to controls, are shown in FIGS. 1A, 1B, 2A, 2B and 3A, 3B. More specifically a placebo formulation (without DMTS), 3-days post dosing, showing a relatively normal respiratory epithelium (FIG. 1A) and a relatively normal olfactory epithelium (FIG. 1B). After 3-days post dosing with formulation F4 herein, the respiratory epithelium did indicate some moderate degeneration and necrosis of the epithelium lining the dorsomedial meatus (FIG. 2A) and some relatively severe unilateral epithelial degeneration, loss and attenuation (FIG. 2B). In addition, after 4 weeks post-dosing of formulation F4, the respiratory epithelium showed relatively mild goblet cell hyperplasia in the middle meatus (FIG. 3A) and the olfactory epithelium indicated unilateral multifocal, relatively minimal degeneration and regeneration of the olfactory epithelium in the dorsomedial meatus. It should be noted that while FIGS. 2A, 2B, 3A and 3B indicated the above referenced relative presence of degeneration as indicated, such damage is contemplated as recoverable damage and less severe than the use of DMTS alone at the indicated concentration. See FIG. 4 which shows use of intranasal delivery of neat DMTS where after 3-days post dosing, there is 100% unilateral loss of olfactory epithelium in all sections examined, along with damage on the contralateral side and multifocal epithelial metaplasia from the respiratory columnar epithelium to pseudostratified squamous epithelium.

Further representative stability studies are included below in Tables 5 and 6. The dimethyl trisulfide formulation evaluated contained 76.5% (wt.) DMTS, 0.3% (wt.) dodecyl maltoside, 12.5% (wt.)ethanol and 10.6% Vitamin E. The DMTS concentration in Table 5 for To (time zero), 1 Month, 3 Months and 6 Months is reported in mg/ml by HPLC. The precision in the measurement of DMTS concentration on a mg/ml basis is +/−3.0%. See Table 6. As can there be observed, the DMTS formulations herein are such that the DMTS, when stored in a sealed ampoule with DMTS at a level of 15.0% (wt.) to 80.0% (wt.), along with maltoside based non-ionic detergent at a level of 0.1% (wt.) to 5.0% (wt.), organic alcohol at a level of 5.0% (wt.) to 65.0% (wt.) and Vitamin E or a Vitamin E derivative at a level of 0.1 wt. % to 50.0 wt. %, is such that the DMTS concentration therein, when stored in a sealed ampoule, provides that the concentration of DMTS therein, over a period of up to 6 months, at a temperature of 2-8 °C, room temperature, or 40 °C, does not vary by more than +/−3.0%.

TABLE 5
Concentration Of DMTS Remaining Stored In Ampoules

| Ampoule | | | | |
|---|---|---|---|---|
| Storage | | | | |
| Conditions | To | 1 Month | 3 Months | 6 Months |
| 2-8 °C | 766 | — | 787 | 789 |
| Room | 766 | — | 778 | 784 |
| Temperature | | | | |
| 40 °C | 766 | 767 | 787 | 772 |

TABLE 6
Percent Of DMTS Remaining Stored In Ampoules

| Ampoule | | | | |
|---|---|---|---|---|
| Storage | | | | |
| Conditions | To | 1 Month | 3 Months | 6 Months |
| 2-8 °C | 100 | — | 103 | 103 |
| Room | 100 | — | 102 | 102 |
| Temperature | | | | |
| 40 °C | 100 | 100 | 103 | 101 |

1. A non-invasive dimethyl trisulfide formulation comprising:
a. dimethyl trisulfide at a level of 15.0% (wt.) to 80.0% (wt.);
b. maltoside based non-ionic detergent at a level of 0.1% (wt.) to 5.0% (wt.);
c. organic alcohol at a level of 5.0% (wt.) to 65.0% (wt.); and
d. Vitamin E or a Vitamin E derivative at a level of 0.1 wt. % to 50.0 wt. %.

2. The non-invasive dimethyl trisulfide formulation of claim 1 wherein said maltoside based non-ionic detergent comprises: 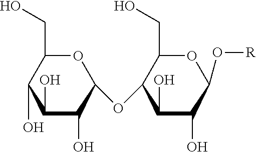
wherein R has 8-15 carbon atoms.

3. The non-invasive dimethyl trisulfide formulation of claim 1 wherein said maltoside based non-ionic detergent comprises at least one of octyl maltoside, nonyl maltoside, undecyl maltoside or dodecyl maltoside.

4. The non-invasive dimethyl trisulfide formulation of claim 1 wherein said organic alcohol is selected from the group consisting of ethanol, propanol, isopropanol, 2-(2-ethoxyethoxy)ethanol, propylene glycol or 2-(2-ethoxyethoxy)ethanol.

5. The non-invasive dimethyl trisulfide formulation of claim 1 further including a poly(ethylene glycol) at a level of 5.0% (wt.) to 40.0% (wt.).

6. The non-invasive dimethyl trisulfide formulation of claim 1 further including water at a level of 5.0% (wt.) to 40.0% (wt.).

7. The non-invasive dimethyl trisulfide formulation of claim 1 further including a glycerol polyethyleneglycol ricinoleate at a level of 5.0% (wt.) to 40.0% (wt.).

8. The non-invasive dimethyl trisulfide formulation of claim 1 further including ethyl laurate at a level of 5.0% (wt.) to 40.0% (wt.).

9. The non-invasive dimethyl trisulfide formulation of claim 1 wherein said dimethyl trisulfide is present at a level of 50.0% (wt.) to 80.0% (wt.)

10. An intranasal delivery device containing a dimethyl trisulfide formulation, wherein said formulation comprises dimethyl trisulfide at a level of 15.0% (wt.) to 80.0% (wt.), maltoside based non-ionic detergent at a level of 0.1% (wt.) to 5.0% (wt.), organic alcohol at a level of 5.0% (wt.) to 65.0% (wt.); and Vitamin E or a Vitamin E derivative at a level of 0.1 wt. % to 50.0 wt. %.

11. The intranasal delivery device of claim 10 wherein said maltoside based non-ionic detergent comprises: 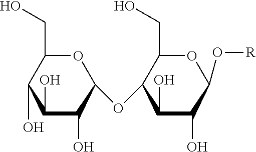
wherein R has 8-15 carbon atoms.

12. The intranasal delivery device of claim 10 wherein said maltoside based non-ionic detergent comprises at least one of octyl maltoside, nonyl maltoside, undecyl maltoside or dodecyl maltoside.

13. The intranasal delivery device of claim 10 wherein said organic alcohol is selected from the group consisting of ethanol, propanol, isopropanol, 2-(2-ethoxyethoxy)ethanol, propylene glycol or 2-(2-ethoxyethoxy)ethanol.

14. The intranasal delivery device of claim 10 further including a poly(ethylene glycol) at a level of 5.0% (wt.) to 40.0% (wt.).

15. The intranasal delivery device of claim 10 further including water at a level of 5.0% (wt.) to 40.0% (wt.).

16. The intranasal delivery device of claim 10 further including a glycerol polyethyleneglycol ricinoleate at a level of 5.0% (wt.) to 40.0% (wt.).

17. The intranasal delivery device of claim 10 further including ethyl laurate at a level of 5.0% (wt.) to 40.0% (wt.).

18. The intranasal delivery device of claim 10 wherein said dimethyl trisulfide is present at a level of 50.0% (wt.) to 80.0% (wt.)

19. A method for intranasal delivery of a dimethyl trisulfide formulation comprising:
a. providing a dimethyl trisulfide formulation comprising dimethyl trisulfide at a level of 15.0% (wt.) to 80.0% (wt.), maltoside based non-ionic detergent at a level of 0.1% (wt.) to 5.0% (wt.), organic alcohol at a level of 5.0% (wt.) to 65.0% (wt.); and Vitamin E or a Vitamin E derivative at a level of 0.1 wt. % to 50.0 wt. %;
b. incorporating said dimethyl trisulfide formulation into an intranasal delivery device; and
c. delivering said dimethyl trisulfide formulation into a nasal cavity.

20. The method of claim 10 wherein said maltoside based non-ionic detergent comprises: 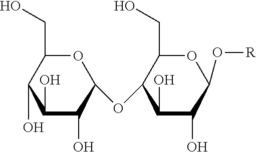
wherein R has 8-15 carbon atoms.
